(19) United States
(12) Patent Application Publication
(10) Pub. No.: US 2018/0070799 A1
(43) Pub. Date: Mar. 15, 2018

(54) IMAGING DEVICE, ENDOSCOPE SYSTEM, AND METHOD OF MANUFACTURING IMAGING DEVICE

(71) Applicant: OLYMPUS CORPORATION, Tokyo (JP)

(72) Inventor: Takanori SEKIDO, Tokyo (JP)

(73) Assignee: OLYMPUS CORPORATION, Tokyo (JP)

(21) Appl. No.: 15/817,455

(22) Filed: Nov. 20, 2017

Related U.S. Application Data
(63) Continuation of application No. PCT/JP2015/065656, filed on May 29, 2015.

(51) Int. Cl. A61B 1/00; A61B 1/04; A61B 1/06; H04N 5/225; H01L 27/146; H01L 23/00

(52) U.S. Cl. CPC A61B 1/0011; A61B 1/04; A61B 1/00045; A61B 1/06; A61B 1/00114; H04N 5/2251; H01L 2224/1713; H01L 24/16; H01L 24/94; H04N 2005/2255; H04N 5/2256; H01L 2224/1623; H01L 27/14636

(57) ABSTRACT

An imaging device includes: an image sensor including a light receiving unit, and sensor electrodes; a relay board including at least one rectangular substrate, one or more electronic components, a body part, substrate electrodes, cable electrodes, and pins standing on the substrate and configured to electrically connect the substrate electrodes and the cable electrodes, wherein the substrate electrodes are electrically and mechanically connected with the sensor electrodes; and a cable assembly including cables, and a cable fixing member, wherein cores of the cables exposed on an connection face of the cable fixing member are electrically and mechanically connected with the cable electrodes. A coefficient of thermal expansion of the image sensor, a coefficient of thermal expansion of the relay board, and a coefficient of thermal expansion of the cable assembly vary gradually in descending order or ascending order.

### CROSS-REFERENCE TO RELATED APPLICATION

This application is a continuation of PCT international application Ser. No. PCT/JP2015/065656 filed on May 29, 2015 which designates the United States, incorporated herein by reference.

### BACKGROUND

The present disclosure relates to an imaging device, an endoscope system with the imaging device, and a method of manufacturing the imaging device.

In the related art, endoscope systems have been widely used for various examinations in the medical and industrial fields. Among them, in a medical endoscope system a long, thin, and flexible insertion part, which has a distal end portion incorporating an imaging device, is inserted into the inside of a subject such as a patient, which allows observation of a site to be examined, or the like. There is a demand for reduction in the diameter of the insertion part of such endoscope system in view of facilitating the introduction into a subject.

An imaging device, which includes an image sensor and a circuit board on which electronic components such as capacitors and IC chips constituting a drive circuit of the image sensor are mounted, is fitted into a distal end of an insertion part of an endoscope used in an endoscope system, and cables are soldered on the circuit board of the imaging device.

In recent years, various imaging devices have been proposed as technologies for facilitating work of connecting signal lines of cables, improving the reliability of connections, and enabling a reduced diameter (refer, for example, to JP 2014-110847 A and JP 2013-118337 A).

### SUMMARY

An imaging device according to one aspect of the present disclosure may include: an image sensor including a light receiving unit configured to perform photoelectric conversion on incident light to generate in electrical signal, and a plurality of sensor electrodes formed on a rear face opposite to a face on which the light receiving unit is formed; a relay board including at least one rectangular substrate, one or more electronic components mounted on the substrate, a body part formed of encapsulating resin encapsulating the electronic components and formed in a quadrangular prism having a cross-section of a same shape as a projection area of the substrate, substrate electrodes formed on a front face of the relay board, cable electrodes formed on a rear face of the relay board, and a plurality of pins standing on the substrate and configured to electrically connect the substrate electrodes and the cable electrodes, wherein the substrate electrodes are electrically and mechanically connected with the sensor electrodes of the image sensor; and a cable assembly including a plurality of cables, and a cable fixing member fixing the cables, wherein cores of the cables exposed on an connection face of the cable fixing member are electrically and mechanically connected with the cable electrodes of the relay board, wherein a coefficient of thermal expansion of the image sensor, a coefficient of thermal expansion of the relay board, and a coefficient of thermal expansion of the cable assembly vary gradually in descending order or ascending order.

The above and other objects, features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view schematically illustrating an overall configuration of an endoscope system according to a first embodiment;

FIG. 2A is a perspective view of an imaging device used in an endoscope in FIG. 1;

FIG. 2B is an exploded perspective view of the imaging device of FIG. 2A;

FIG. 3 is a cross-sectional view of the imaging device of FIGS. 2A and 2B;

FIG. 4A is a view explaining a method of manufacturing the imaging device according to the first embodiment;

FIG. 4B is a view explaining the method of manufacturing the imaging device according to the first embodiment;

FIG. 4C is a view explaining the method of manufacturing the imaging device according to the first embodiment;

FIG. 4D is a view explaining the method of manufacturing the imaging device according to the first embodiment;

FIG. 4E is a view explaining the method of manufacturing the imaging device according to the first embodiment;

FIG. 5 is a cross-sectional view of an imaging device according to a first modification of the first embodiment;

FIG. 6 is a cross-sectional view of an imaging device according to a second modification of the first embodiment;

FIG. 7 is a perspective view of a relay board according to the second embodiment;

FIG. 8 is a perspective view of a relay board according to a first modification of the second embodiment;

FIG. 9 is a perspective view of a relay board according to a second modification of the second embodiment;

FIG. 10 is a perspective view of a relay board according to a third modification of the second embodiment;

FIG. 11 is a perspective view of a relay board according to a fourth modification of the second embodiment;

FIG. 12 is a perspective view of a relay board according to a fifth modification of the second embodiment;

FIG. 13 is a perspective view of a relay board according to a third embodiment; and

FIG. 14 is a perspective view of a relay board according to a first modification of the third embodiment.

### DETAILED DESCRIPTION

In the following description, an endoscope device with an imaging module, which is a mode for carrying out the present disclosure (hereinafter referred to as an “embodiment”), will be described. Note that the present disclosure is not limited to the embodiment. In depiction of the drawings, the same components will be designated by the same reference numerals. Furthermore, note that the drawings are schematic, and that the relations between the thicknesses and the widths of respective members, the ratios of the members and the like may be different from the actual relations, ratios, and the like. Furthermore, a drawing may also include parts with dimensions and ratios different from those of another drawing.

### First Embodiment

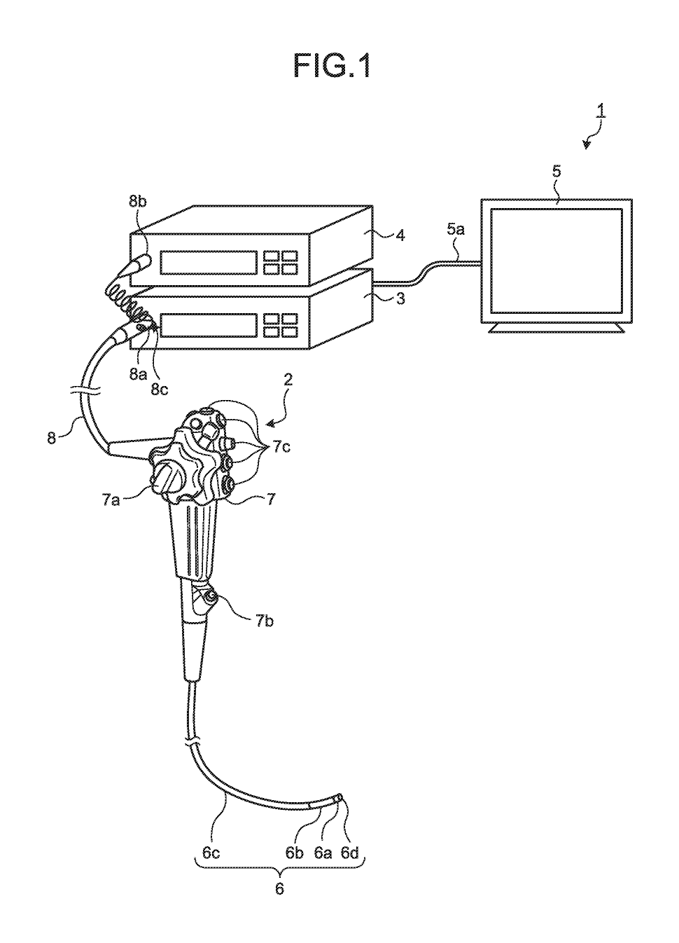

FIG. 1 is a view schematically illustrating an overall configuration of an endoscope system according to an embodiment of the present disclosure. As illustrated in FIG. 1, an endoscope system 1 according to the first embodiment includes an endoscope 2 to be inserted into a subject and being configured to image the inside of the body of the subject and generate an image of the inside of the subject, an information processing device 3 (external processor) configured to perform predetermined image processing on the image signal captured by the endoscope 2 and control the respective components of the endoscope system 1, a light source device 4 configured to generate illumination light for the endoscope 2, and a display device 5 configured to display an image corresponding to the image signal resulting from image processing performed by the information processing device 3.

The endoscope 2 includes an insertion part 6 to be inserted into a subject, an operating unit 7, which is provided adjacent to a proximal end portion of the insertion part 6 and which is to be gripped by an operator, and flexible universal cord 8 extending from the operating unit 7.

The insertion part 6 is constituted by an illumination fiber (light guide cable), an electric cable, an optical fiber, and the like. The insertion part 6 has a distal end portion 6a incorporating an imaging unit, which will be described later, a bending portion 6b, which is freely bendable and constituted by a plurality of bending pieces, and a flexible tube portion 6c, which is flexible and provided on the proximal end side of the bending portion 6b. The distal end portion 6a is provided with an illumination portion for illuminating the inside of a subject through an illumination lens, an observation portion for capturing the inside of the subject, an aperture through which a channel for a treatment tool communicates, and an air/water nozzle (not illustrated).

The operating unit 7 includes a bending nob 7a for bending the bending portion 6b upward, downward, leftward, and rightward, a treatment tool insertion part 7b through which a treatment tool such as living body forceps or a laser knife is inserted into the body cavity of a subject, and a plurality of switches 7c for operation of peripheral devices such as the information processing device 3, the light source device 4, an air conveyance device, a water conveyance device, and a gas conveyance device. A treatment tool inserted through the treatment tool insertion part 7b passes through a treatment tool channel formed thereinside and comes out from an aperture 6d at the distal end of the insertion part 6.

The universal cord 8 is constituted by an illumination fiber, a cable, and the like. The universal cord 8 branches at the proximal end into one end being a connector 8a and the other proximal end being a connector 8b. The connector 8a is removably connected with a connector of the information processing device 3. The connector 8b is removably connected with the light source device 4. The universal cord 8 propagates illumination light emitted by the light source device 4 to the distal end portion 6a through the connector 8b and the illumination fiber. The universal cord 8 also transmits an image signal captured by the imaging device, which will be described below, to the information processing device 3 through the cable and the connector 8a.

The information processing device 3 performs predetermined image processing on an image signal output from the connector 8a, and controls the whole endoscope system 1.

The light source device 4 is constituted by a light source that emits light, a condenser lens, and the like. The light source device 4 emits light from the light source, and supplies the light as light for illuminating the inside of a subject to be imaged to the endoscope 2 connected via the connector 8b and the illumination fiber of the universal cord 8 under the control of the information processing device 3.

The display device 5 is constituted by a display of liquid crystal or organic electro-luminescence (EL), or the like. The display device 5 displays various information data, including images subjected to the predetermined image processing performed by the information processing device 3, via a video cable 5a. This allows the operator to operate the endoscope 2 while looking at an image (in-vivo image) displayed by the display device 5 to observe a desired position in the subject and determine characteristics.

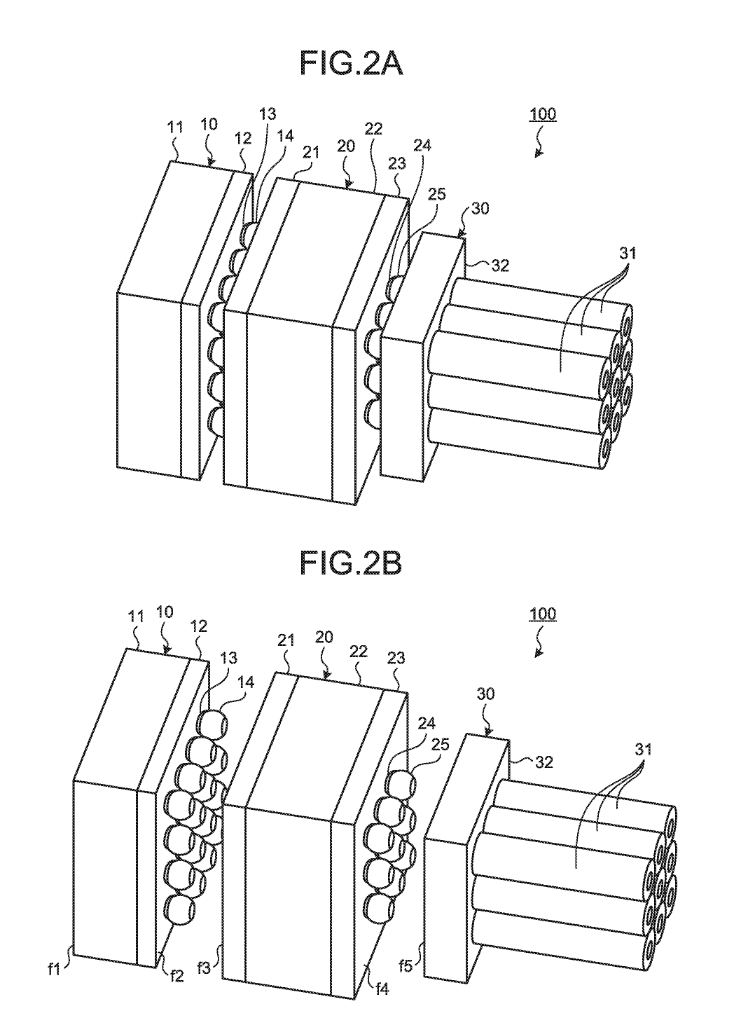

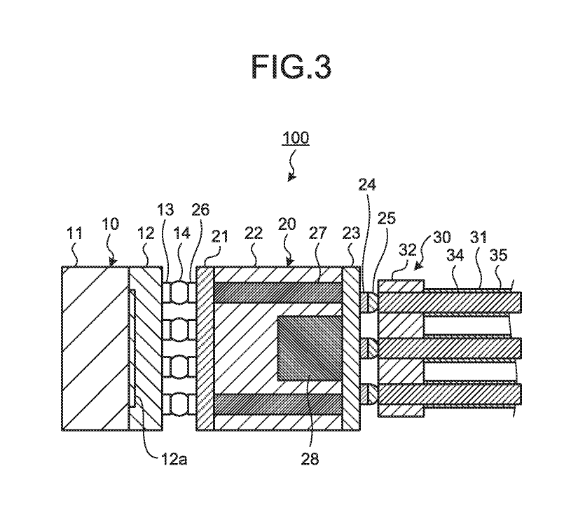

Next, a configuration of an imaging device will be described in detail. FIG. 2A is a perspective view of an imaging device used in the endoscope in FIG. 1. FIG. 2B is an exploded perspective view of the imaging device of FIG. 2A. FIG. 3 is a cross-sectional view of the imaging device of FIGS. 2A and 2B.

As illustrated in FIG. 2, an imaging device 100 according to the first embodiment includes an image sensor 10, a relay board 20, and a cable assembly 30.

The image sensor 10 has a structure in which glass 11 is adhered to an image sensor chip 12. Light collected by a lens unit is incident on a light receiving surface of the image sensor chip 12 having a light receiving unit 12a via an f1 face that is a front face of the glass 11. Bumps 14 each made of a sensor electrode 13, solder, and the like are formed on an f2 face (rear face) of the image sensor 10. The image sensor 10 is preferably a chip size package (CSP) obtained by performing wiring, electrode formation, resin encapsulation, and dicing on the image sensor chip 12 in the form of a wafer, and eventually having a package size corresponding to the size of the image sensor chip 12.

The relay board 20 has two substrates 21 and 23 having the same rectangular shape, an electronic component 28 mounted on the substrate 23, a body part 22 made of encapsulating resin encapsulating the electronic component 28 and formed into a rectangular shape that is the same shape as a projection area of the substrates 21 and 23, substrate electrodes 26 formed on an f3 face that is a front face, cable electrodes 24 formed on an f4 face that is a rear face, and a plurality of pins 27 standing on the substrate 23 and electrically connecting the substrate electrodes 26 and the cable electrodes 24. The relay board 20 has a function of rewiring by changing the positions at which the substrate electrodes 26 and the cable electrodes 24 are arranged on a plane perpendicular to the optical axis direction. Alternatively, the positions at which the substrate electrodes 26 and the cable electrodes 24 are arranged on a plane perpendicular to the optical axis direction may be the same and the arrangement may be changed by wiring on the substrate 21 or 23. The relay board 20 is positioned such that the substrates 21 and 23 are perpendicular to the optical axis of the image sensor 10 (parallel to the image sensor 10), and the substrate electrodes 26 are electrically and mechanically connected with the sensor electrodes 13 of the image sensor 10 with the bumps 14 therebetween.

For the encapsulating resin of the body part 22, insulating thermosetting resin or ultraviolet curable resin is used. Vias or through-electrodes, or wiring, which are not illustrated, are formed on the substrates 21 and 23 and connected with the pins 27. The electronic component 28 may be mounted on the substrate 21. While the connection with the image sensor 10 is provided by the substrate electrodes 26 formed on the substrate 21 in the first embodiment, the connection with the image sensor 10 may be provided by pins 27 exposed on an end face or substrate electrodes 26 formed on the pins 27 without use of the substrate 21. Alternatively, the electronic component 28 and the pins 27 may be connected with the substrate 21, and the connection with the image sensor 10 may be provided by pins 27 exposed on an end face or cable electrodes 24 formed on the pins 27 without use of the substrate 23. Although not illustrated, at the connections between the sensor electrodes 13 and the substrate electrodes 26 are filled with encapsulating resin for the purpose of improving the reliability of the connections.

The cable assembly 30 includes nine cables 31 and a cable fixing member 32 for fixing the cables 31.

The cables 31 are each constituted by a conductive core 34 and an insulator 35 coating the circumference of the core 34. At distal end of the cables 31, the insulators 35 are stripped off at a predetermined length such that the cores 34 expose.

The cable fixing member 32 is for fixing the exposed cores 34, and insulating thermosetting resin or ultraviolet curable resin is used therefor. End faces of the cores 34 of the cables 31 are exposed on an f5 face, which is a connecting face of the cable fixing member 32, and the exposed core ends are electrically and mechanically connected with the cable electrodes 24 of the relay board 20 with a bumps 25 therebetween. Although not illustrated, at the connections between the cable electrodes 24 and the ends of the cores 34 of the cables 31 are filed with encapsulating resin for the purpose of improving the reliability of the connections.

As described above, the connections between the sensor electrodes 13 and the substrate electrodes and the connections between the cable electrodes 24 and the ends of the cores 34 of the cables 31 are encapsulated with encapsulating resin so that disconnection of the connections is prevented; if, however, the environmental temperature increases owing to heat generation of the image sensor 10, the electronic component, or the like, a great thermal stress may be applied to a connection owing to a difference in coefficients of thermal expansion of materials used for the image sensor 10, the relay board 20, and the cable assembly 30, which may cause a crack or disconnection at the connection. In the first embodiment, in order to reduce thermal stress applied to the connections, the materials are selected so that the coefficient of thermal expansion of the image sensor 10, the coefficient of thermal expansion of the relay board 20, and the coefficient of thermal expansion of the cable assembly 30 are in descending order or ascending order. The coefficient of thermal expansion of the encapsulating resin forming the body part 22 contributes most to the coefficient of thermal expansion of the relay board 20, and the resin forming the cable fixing member 32 contributes most to the coefficient of thermal expansion of the cable assembly 30. Thus, the materials may be selected so that the coefficient of thermal expansion of the image sensor 10, the coefficient of thermal expansion of the encapsulating resin forming the body part 22, and the coefficient of thermal expansion of the resin forming the cable fixing member 32 are in descending order or ascending order. Note that, since the coefficients of thermal expansion of the respective members arranged continuously only need to gradually vary so that the coefficients of thermal expansion of adjacent materials do not differ greatly from each other in order to reduce thermal stress applied to the connections, the coefficient of thermal expansion α1 of the image sensor 10, the coefficient of thermal expansion α2 of the encapsulating resin forming the body part 22, and the coefficient of thermal expansion α3 of the resin forming the cable fixing member 32 only need to satisfy α1≧α2≧α3 or α1≦α2≦α3, for example. Thus, α1=α2=α3 may be included, and α1>α2=α3, α1=α2>α3, and the like, which satisfy the above expression are included. Such variation in the coefficient of thermal expansion of the respective members arranged continuously will be referred to as “gradual variation” in the description below.

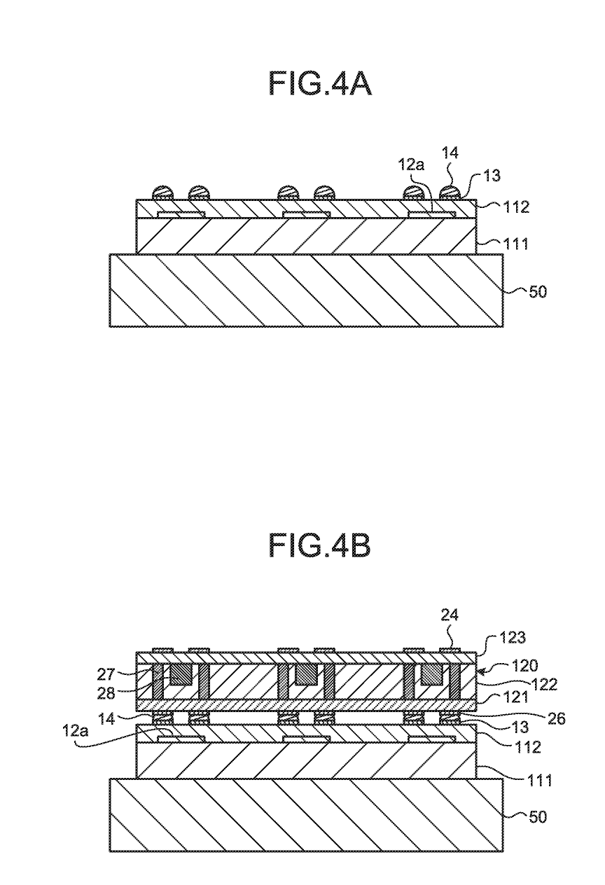

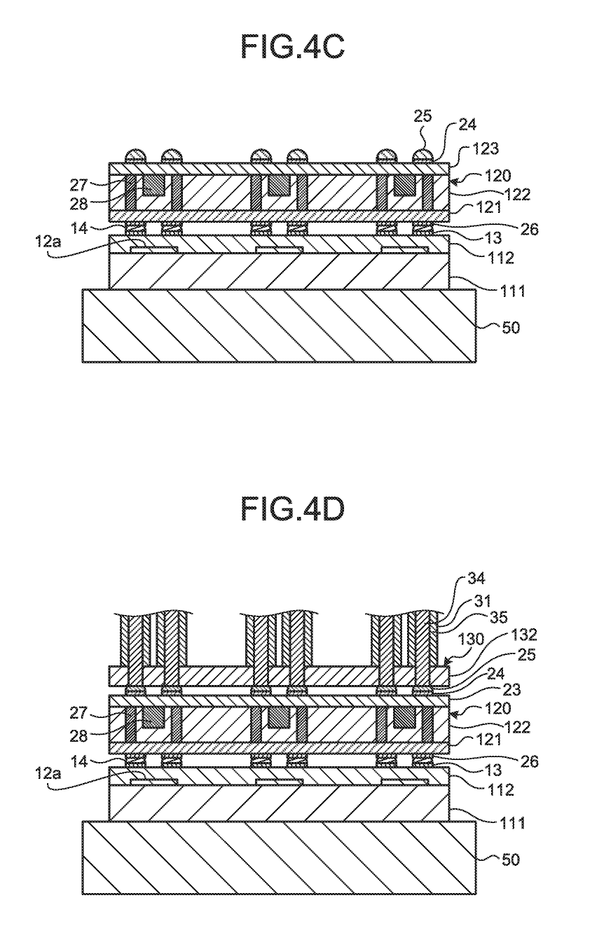

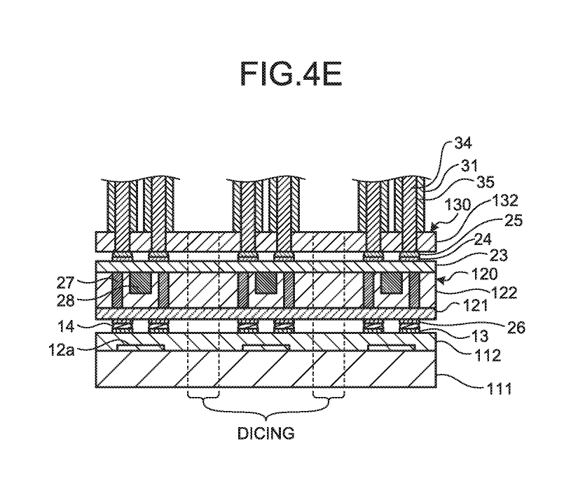

Next, a method of manufacturing the imaging device 100 will be explained with reference to the drawings. FIGS. 4A to 4E are views explaining the method of manufacturing the imaging device 100 according to the first embodiment. Note that, in FIGS. 4A to 4E, the drawings are simplified by illustrating reduced numbers of sensor electrodes 13, cable electrodes 24, cables 31, and the like.

First, as illustrated in FIG. 4A, a wafer 112 which is connected with a glass plate 111 and on which a plurality of light receiving units 12a and sensor electrodes 13 are formed is placed on a stage 50, and bumps 14 are formed on the sensor electrodes 13.

A plurality of electronic components 28 and pins 27 are mounted on the rear face side of a substrate 123 on which a plurality of cable electrodes 24 are formed, the electronic components 28 and the pins 27 are encapsulated with encapsulating resin to form a body part 122, and the side of the body part 122 at which the ends of the pins 27 expose and a substrate 121 are connected with each other, so that a mother board 120 of the relay board 20 is produced (see FIG. 4B).

Thereafter, the mother board 120 is placed as illustrated in FIG. 4B on the wafer 112 on which the bumps 14 are formed as illustrated in FIG. 4A, and the sensor electrodes 13 and the substrate electrodes 26 are connected with each other by the bumps 14 (first connection step).

A large cable assembly 130 in which plurality of cables 31 having ends at which insulators 35 are stripped off are fixed by a cable fixing member 132 is produced (see FIG. 4D).

Bumps 25 are formed on the cable electrodes 24 as illustrated in FIG. 4C, and the cores 34 of the cables 31 exposed on the connection face of the large cable assembly 130 connected by the cable fixing member 132 and the cable electrodes 24 formed on the rear face of the mother board 120 are connected with the bumps 25 therebetween as illustrated in FIG. 4D (second connection step).

After the wafer 112, the mother board 120, and the large cable assembly 130 are connected, dicing is performed at positions indicated by dotted lines in FIG. 4E so that individual imaging devices 100 are obtained.

Manufacture as described above allows a large number of imaging devices 100 to be produced at lower costs. Note that an individual image sensor 10 obtained by dicing, a relay board 20 and a cable assembly 30 may be connected to produce an imaging device 100, but the connection is preferably such that the cable assembly 30 and the relay board 20 are connected with each other and the image sensor 10 is then connected with the relay board 20. The connection in this order reduces disposal costs when failure occurs in connections.

In the first embodiment, since the relay board 20 and the cable assembly 30 are within a projection area in the optical axis direction of the image sensor 10 and the cable assembly 30 is in end-to-end connection with the relay board 20, the imaging device 100 is miniaturized. In addition, since the coefficients of thermal expansion of the image sensor 10, the relay board 20, and the cable assembly 30 vary gradually in descending order or in ascending order, thermal stress applied to the connections between the image sensor 10, the relay board 20, and the cable assembly 30 is reduced, which improves the reliability of the connections.

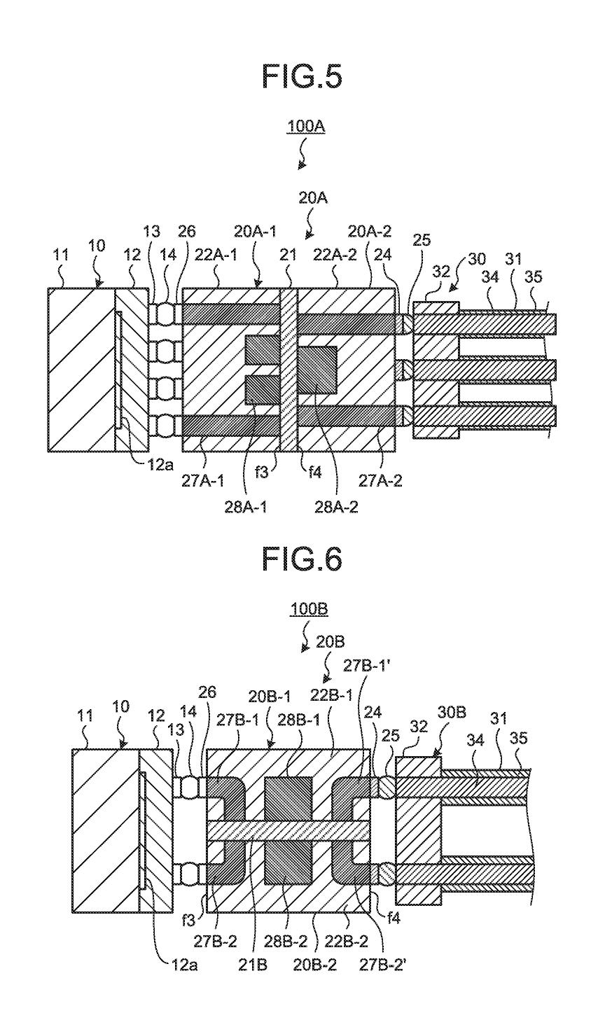

Note that while the relay board 20 in the first embodiment described above includes two substrates 21 and 23, a relay board may include body parts provided on respective faces of one substrate. FIG. 5 is a cross-sectional view of an imaging device according to a first modification of the first embodiment.

In an imaging device 100A according to the first modification of the first embodiment, a first relay unit 20A-1 and a second relay unit 20A-2 are formed on an f3 face and an f4 face, respectively, of a substrate 21 of a relay board 20A. The first relay unit 20A-1 includes an electronic component 28A-1 mounted on the f3 face of the substrate 21, a plurality of first pins 27A-1 standing on the f3 face of the substrate 21, a first body part 22A-1 formed of first encapsulating resin encapsulating the electronic component 28A-1 and the first pins 27A-1, and substrate electrodes 26 formed on the front face of the first body part 22A-1. The second relay unit 20A-2 includes an electronic component 28A-2 mounted on the f4 face of the substrate 21, a plurality of second pins 27A-2 standing on the f4 face of the substrate 21, a second body part 22A-2 formed of second encapsulating resin encapsulating the electronic component 28A-2 and the second pins 27A-2, and cable electrodes 24 formed on the rear face of the second body part 22A-2. The electronic component 28A-1 and the electronic component 28A-2 may be the same components or different components.

In the imaging device 100A, the materials are preferably selected such that the coefficient of thermal expansion of the image sensor 10, the coefficient of thermal expansion of the first encapsulating resin forming the first body part 22A-1, the coefficient of thermal expansion of the second encapsulating resin forming the second body part 22A-2, and the coefficient of thermal expansion of the cable assembly 30 (the resin forming the cable fixing member 32) gradually vary in descending order or in ascending order. With the first modification of the first embodiment, more electronic components may be mounted, and the reliability of the connections are further improved. Note that, for further increasing the number of electronic components mounted, two substrates may be used and three body parts may be formed.

Furthermore, a relay board may be positioned such that a substrate is parallel to the optical axis of the image sensor 10. FIG. 6 is a cross-sectional view of an imaging device according to a second modification of the first embodiment.

In an imaging device 100B according to the second modification of the first embodiment, a substrate 21B of a relay board 20B is positioned to be parallel to the optical axis of the image sensor 10. A first relay unit 20B-1 and a second relay unit 20B-2 are formed on an upper face and a lower face, respectively of the relay board 20B. The first relay unit 20B-1 includes an electronic component 28B-1 mounted on the upper face of the substrate 21B, a plurality of first pins 27B-1 standing on the upper face of the substrate 21B, being bent at bending portions, and having end faces exposed at the f3 face, a plurality of first pins 27B-1′ standing on the upper face of the substrate 21B, being bent at bending portions, and having end faces exposed at the f4 face, and a first body part 22B-1 formed of first encapsulating resin encapsulating the electronic component 28B-1, the first pins 27B-1, and the first pins 27B-1′. The second relay unit 20B-2 includes an electronic component 28B-2 mounted on the lower face of the substrate 21B, a plurality of second pins 27B-2 standing on the lower face of the substrate 21B, being bent at bending portions, and having end faces exposed at the f3 face, a plurality of second pins 27B-2′ standing on the lower face of the substrate 21B, being bent at bending portions, and having end faces exposed at the f4 face, and a second body part 22B-2 formed of second encapsulating resin encapsulating the electronic component 28B-2, the second pins 28B-1, and the second pins 28B-1′. The first encapsulating resin forming the first body part 22B-1 and the second encapsulating resin forming the second body part 22B-2 preferably have coefficients of thermal expansion of comparative levels, and are more preferably the same encapsulating resin.

Substrate electrodes 26 are formed on the ends of the first pins 27B-1 and the second pins 27B-2 exposed on the f3 face, and connected with the sensor electrodes 13 with bumps 14 therebetween. Cable electrodes 24 are formed on the ends of the first pins 27B-1′ and the second pins 27B-2′ exposed on the f4 face, and connected with the cores 34 of the cables 31 with bumps 25 therebetween.

In the imaging device 100B, since the materials are selected such that the coefficient of thermal expansion of the image sensor 10, the coefficient of thermal expansion of the first encapsulating resin forming the first body part 22B-1 (the coefficient of thermal expansion of the second encapsulating resin forming the second body part 22B-2), and the coefficient of thermal expansion of the cable assembly 30 (the resin forming the cable fixing member 32) gradually vary in descending order or in ascending order, the reliability of the connections is improved. Furthermore, in the second modification of the first embodiment, more electronic components may be mounted.

### Second Embodiment

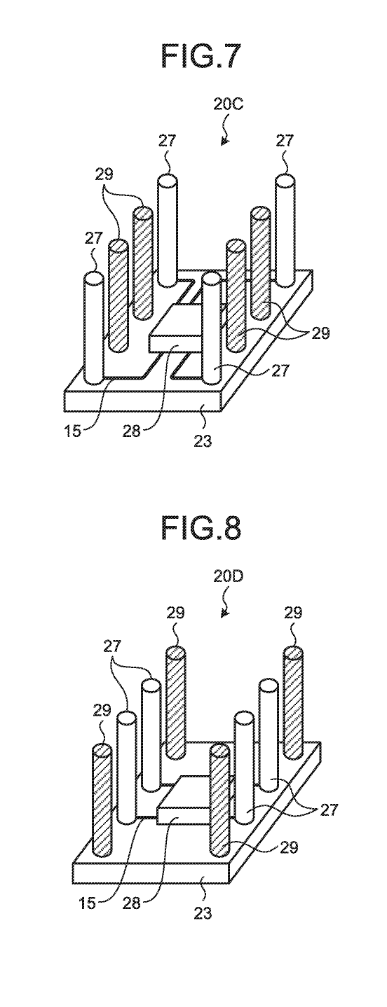

In a second embodiment, a plurality of dummy pins that are not electrically connected with the sensor electrodes and/or the cores are provided on a substrate of a relay board. FIG. 7 is a perspective view of a relay board according to the second embodiment. In FIG. 7, for easier understanding of the disclosure, encapsulating resin forming a body part and the substrate 21 on the image sensor 10 side are not illustrated. Furthermore, the relay board described below is connected with the image sensor 10 and the cable assembly 30 and used as an imaging device similarly to the relay board of the first embodiment.

In a relay board 20C, pins 27 stand at four corners of a substrate 23, and an electronic component 28 is mounted at the center of the substrate 23. The pins 27 and the electronic component 28 are connected by wiring 15. A plurality of dummy pins 29 that are not electrically connected with the sensor electrodes and/or the cores stand along long sides of the substrate 23 and symmetrically with respect to the electronic component 28. The dummy pins 29 are made of the same material as the pins 27, that is, a metal material being excellent in electrical conductivity and thermal conductivity and having a greater stiffness than the encapsulating resin forming the body part of the relay board 20C.

As a result of providing the dummy pins 29 standing in the relay board 20C as in the second embodiment, the relay board 20C is improved in stiffness and functions as a filler, which prevents warping or the like when a thermal load is applied and improves the reliability of the connections.

Alternatively, dummy pins may stand at positions where stress on a relay board concentrates, and pins electrically and mechanically connected with the sensor electrodes and/or the cores may stand avoiding the positions where stress concentrates. FIG. 8 is a perspective view of a relay board according to a first modification of the second embodiment. In FIG. 8, for easier understanding of the disclosure, encapsulating resin forming a body part and the substrate 21 on the image sensor 10 side are not illustrated. Furthermore, the relay board described below is connected with the image sensor 10 and the cable assembly 30 and used as an imaging device similarly to the relay board of the first embodiment.

In a relay board 20D, dummy pins 29 stand at four corners of the substrate 23 where stress concentrates. Pins 27 stand along the long sides of the substrate 23 where stress does not concentrate, and connected with an electronic component 28 mounted at the center of the substrate 23 via wiring 15.

In the first modification of the second embodiment, since the dummy pins 29 stand at positions where stress concentrates within the relay board 20D, the relay board 20D is improved in stiffness, and since the pins 27 stand at positions where stress does not concentrate, the reliability of the connections is improved. In addition, since the pins and the dummy pins function as fillers, warping or the like when a thermal load is applied is prevented and the reliability of the connections is further improved.

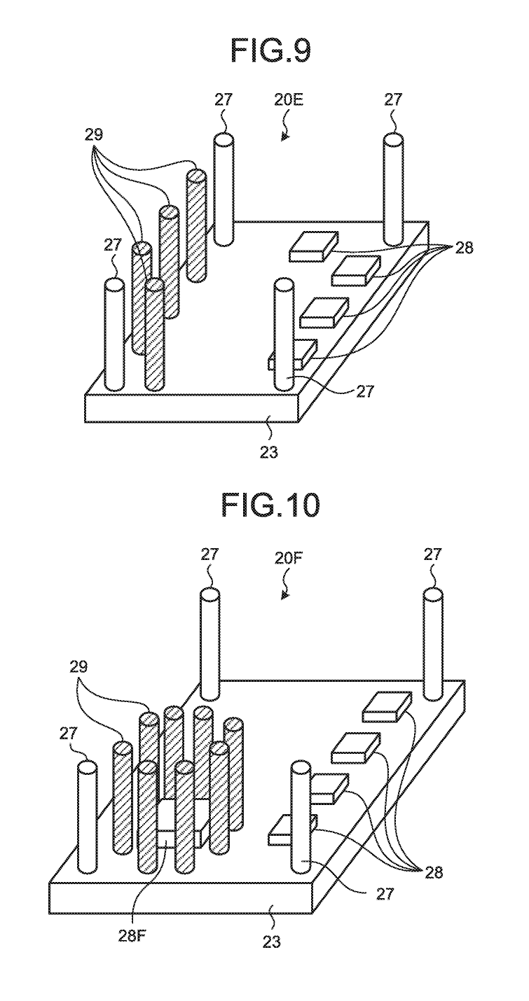

Furthermore, if the position at which an electronic component is mounted on a substrate is biased, dummy pins are preferably arranged at positions where stress concentrates owing to the biased mounting position of the electronic component. FIG. 9 is a perspective view of a relay board according to a second modification of the second embodiment. In FIG. 9, for easier understanding of the disclosure, encapsulating resin forming a body part and the substrate 21 on the image sensor 10 side are not illustrated. Furthermore, the relay board described below is connected with the image sensor 10 and the cable assembly 30 and used as an imaging device similarly to the relay board of the first embodiment.

In a relay board 20E, pins 27 stand at four corners of the substrate 23, and a plurality of electronic components 28 are mounted at positions on one side of the substrate 23. In addition, dummy pins 29 stand at positions symmetrical to the electronic components 28 with respect to the center of the substrate 23.

In the second modification of the second embodiment, since the dummy pins stand at positions symmetrical to the electronic components 28, stress is balanced within the relay board 20E and the stiffness is increased. In addition, since the pins and the dummy pins function as fillers, warping or the like when a thermal load is applied is prevented and the reliability of the connections is improved.

Furthermore, when an electronic component having a high heat radiation is mounted on the substrate 23, dummy pins 29 may stand around the electronic component to release heat. FIG. 10 is a perspective view of a relay board according to a third modification of the second embodiment. In FIG. 10, for easier understanding of the disclosure, encapsulating resin forming a body part and the substrate 21 on the image sensor 10 side are not illustrated. Furthermore, the relay board described below is connected with the image sensor 10 and the cable assembly 30 and used as an imaging device similarly to the relay board of the first embodiment.

In a relay board 20F, pins 27 stand at four corners of the substrate 23, and a plurality of electronic components 28 and an electronic component 28F having a higher heat radiation than the electronic components 28 are mounted. A plurality of dummy pins 29 that are not electrically connected with the sensor electrodes and/or the cores stand around the electronic component 28F.

The dummy pins 29 are made of the same material as the pins 27, that is, a metal material being excellent in electrical conductivity and thermal conductivity and having a greater stiffness and a higher thermal conductivity than the encapsulating resin forming the body part of the relay board 20F. Since the dummy pins 29 excellent in thermal conductivity are arranged around the electronic component 28F, heat radiated by the electronic component 28F is released to outside of the relay board 20F by the dummy pins 29. This reduces the load of thermal stress on the relay board 20F and improves the reliability of the connections.

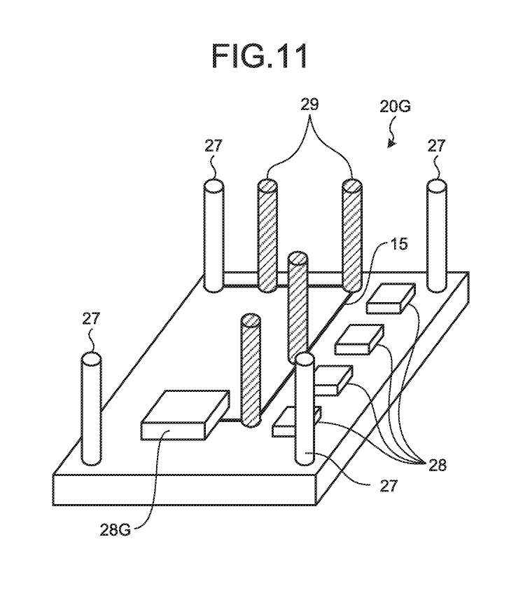

Furthermore, when an electronic component having a high heat radiation is mounted on the substrate 23, dummy pins 29 may be arranged on the wiring 15 drawn from the electronic component. FIG. 11 is a perspective view of a relay board according to a fourth modification of the second embodiment. In FIG. 11, for easier understanding of the disclosure, encapsulating resin forming a body part and the substrate 21 on the image sensor 10 side are not illustrated. Furthermore, the relay board described below is connected with the image sensor 10 and the cable assembly 30 and used as an imaging device similarly to the relay board of the first embodiment.

In a relay board 20G, pins 27 stand at four corners of the substrate 23, and a plurality of electronic components 28 and an electronic component 28G having a higher heat radiation than the electronic components 28 are mounted. In addition, dummy pins 29 stand on the wiring 15 drawn from the electronic component 28G.

Since the dummy pins 29 excellent in thermal conductivity are arranged on the wiring 15 drawn from the electronic component 28G, heat radiated by the electronic component 28G is released to outside of the relay board 20G by the dummy pins 29 via the wiring 15. This reduces the load of thermal stress on the relay board 20G and improves the reliability of the connections. In addition, the stiffness of the dummy pins 29 improves the stiffness of the relay board 20G.

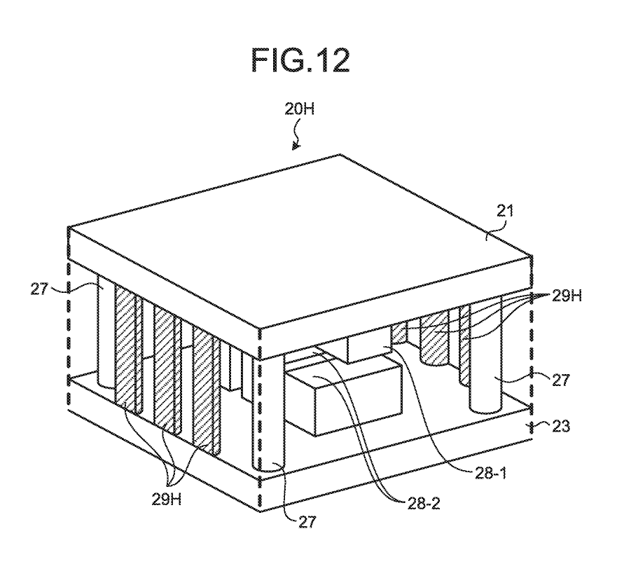

Furthermore, dummy pins may stand to be exposed on a side face, which is parallel to the optical axis direction, of the body part. FIG. 12 is a perspective view of a relay board according to a fifth modification of the second embodiment. In FIG. 12, for easier understanding of the disclosure, encapsulating resin forming a body part and substrate electrodes on the substrate 21 are not illustrated. Furthermore, the relay board described below is connected with the image sensor 10 and the cable assembly 30 and used as an imaging device similarly to the relay board of the first embodiment.

In a relay board 20H, pins 27 stand at four corners of the substrate 23, and a plurality of electronic components 28-2 are mounted. In addition, an electronic component 28-1 is mounted on the substrate 21. Dummy pins 29H stand to be exposed on two side faces, which are opposite to each other and parallel to the optical axis direction, of the body part of the relay board 20H. The dummy pins 29H each have a hemispherical shape; however, the shape of the dummy pins 29H is not limited thereto, and may be such that one side face of a quadrangular prism is exposed.

Since the dummy pins 29H excellent in thermal conductivity are arranged to be exposed on a side face of the body part, heat radiated by the electronic components 28-1 and 28-2 is released to the side face side of the relay board 20H more efficiently. This reduces the load of thermal stress on the relay board 20H, and improves the reliability of the connections. In addition, the stiffness of the dummy pins 29H improves the stiffness of the relay board 20H.

### Third Embodiment

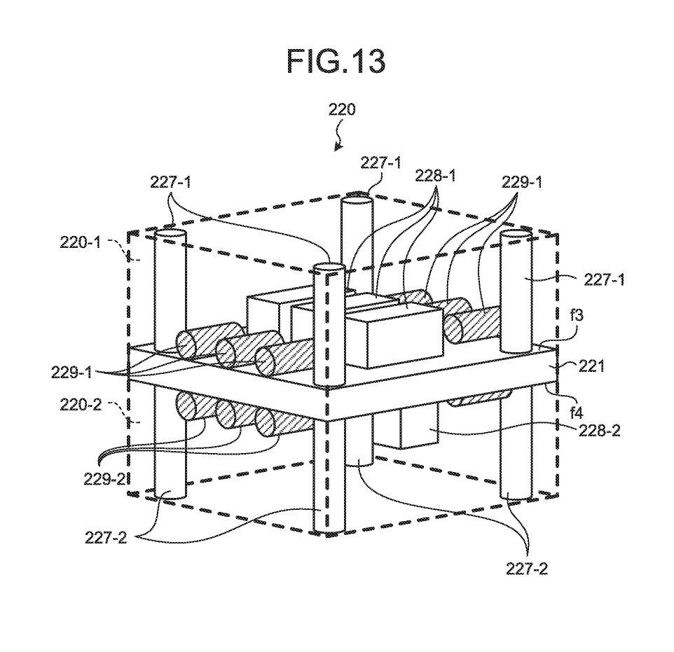

In a third embodiment, dummy pins are arranged horizontally to be exposed to a side face, which is parallel to the optical axis direction, of the body part. FIG. 13 is a perspective view of a relay board according to the third embodiment. In FIG. 13, for easier understanding of the disclosure, encapsulating resin forming a first body part and a second body part is not illustrated. Furthermore, the relay board described below is connected with the image sensor 10 and the cable assembly 30 and used as an imaging device similarly to the relay board of the first embodiment.

In a relay board 220 of the third embodiment, a first relay unit 220-1 and a second relay unit 220-2 are formed on an f3 face and an f4 face, respectively, of a substrate 221. In the first relay unit 220-1, electronic components 228-1 are mounted and a plurality of first pins 227-1 stand on the f3 face of the substrate 221. In addition, a plurality of dummy pins 229-1 are arranged horizontally at opposed positions with the electronic components 228-1 therebetween. In the second relay unit 220-2, electronic components 228-2 are mounted and a plurality of second pins 227-2 stand on the f4 face of the substrate 221. In addition, a plurality of dummy pins 229-2 are arranged horizontally at opposed positions with the electronic components 228-2 therebetween. The end faces of the dummy pins 229-1 and 229-2 are exposed at side faces, which are parallel to the optical axis direction, of the first body part and the second body part.

In the third embodiment, since the dummy pins 229-1 and 229-2 excellent in thermal conductivity are arranged horizontally to be exposed on side faces of the first body part and the second body part, heat radiated by the electronic components 228-1 and 228-2 is released to the side face sides of the relay board 220 more efficiently. This reduces the load of thermal stress on the relay board 220, and improves the reliability of the connections.

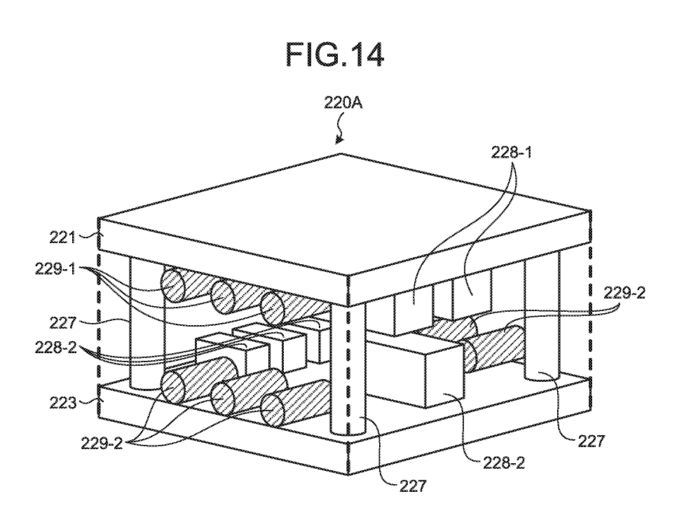

Furthermore, in a relay board including two substrates, dummy pins may also be arranged horizontally to release heat toward side faces of the relay board. FIG. 14 is a perspective view of a relay board according to a first modification of the third embodiment. In FIG. 14, for easier understanding of the disclosure, encapsulating resin forming a body part and substrate electrodes formed on a substrate 221 are not illustrated. Furthermore, the relay board described below is connected with the image sensor 10 and the cable assembly 30 and used as an imaging device similarly to the relay board of the first embodiment.

In a relay board 220A according to the first modification of the third embodiment, a plurality of electronic components 228-1 are mounted on the substrate 221, and a plurality of electronic components 228-2 are mounted on a substrate 223. A plurality of pins 227 are arranged between the substrates 221 and 223 opposed to each other. In addition, a plurality of dummy pins 229-1 are arranged horizontally at opposed positions with the electronic components 228-1 therebetween, and a plurality of dummy pins 229-2 are arranged horizontally at opposed positions with the electronic components 228-2 therebetween. The end faces of the dummy pins 229-1 and 229-2 are exposed at side faces, which are parallel to the optical axis direction, of the body part.

In the first modification of the third embodiment, since the dummy pins 229-1 and 229-2 excellent in thermal conductivity are arranged horizontally to be exposed on side faces of the body part, heat radiated by the electronic components 228-1 and 228-2 is released to the side face sides of the relay board 220A more efficiently. This reduces the load of thermal stress on the relay board 220A, and improves the reliability of the connections.

According to the present disclosure, a miniaturized imaging device having high reliability of connections is obtained.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging device comprising:
an image sensor including
a light receiving unit configured to perform photoelectric conversion on incident light to generate in electrical signal, and
a plurality of sensor electrodes formed on a rear face opposite to a face on which the light receiving unit is formed;
a relay board including
at least one rectangular substrate,
one or more electronic components mounted on the substrate,
a body part formed of encapsulating resin encapsulating the electronic components and formed in a quadrangular prism having a cross-section of a same shape as a projection area of the substrate,
substrate electrodes formed on a front face of the relay board,
cable electrodes formed on a rear face of the relay board, and
a plurality of pins standing on the substrate and configured to electrically connect the substrate electrodes and the cable electrodes,
wherein the substrate electrodes are electrically and mechanically connected with the sensor electrodes of the image sensor; and
a cable assembly including
a plurality of cables, and
a cable fixing member fixing the cables,
wherein cores of the cables exposed on an connection face of the cable fixing member are electrically and mechanically connected with the cable electrodes of the relay board,
wherein a coefficient of thermal expansion of the image sensor, a coefficient of thermal expansion of the relay board, and a coefficient of thermal expansion of the cable assembly vary gradually in descending order or ascending order.

2. The imaging device according to claim 1,
wherein the substrate of the relay board is positioned to be perpendicular to an optical axis of the image sensor, and a first relay unit and a second relay unit are formed on a front face and a rear face, respectively, of the substrate,
the first relay unit includes
a first electronic component mounted on the front face of the substrate,
a first body part formed of first encapsulating resin encapsulating the first electronic component,
the substrate electrodes provided on a front face of the first body part, and
a plurality of first pins standing on the front face of the substrate,
the second relay unit includes
a second electronic component mounted on the rear face of the substrate,
a second body part formed of second encapsulating resin encapsulating the second electronic component,
the cable electrodes provided on a rear face of the second body part, and
a plurality of second pins standing on the rear face of the substrate, and
the coefficient of thermal expansion of the image sensor, a coefficient of thermal expansion of the first encapsulating resin, a coefficient of thermal expansion of the second encapsulating resin, and the coefficient of thermal expansion of the cable assembly vary gradually in descending order or ascending order.

3. The imaging device according to claim 2, wherein
the relay board and the cable assembly have sizes that are equal to or smaller than a projection area of the image sensor in an optical axis direction, and
positions of the substrate electrodes and positions from the cable electrodes on a plane perpendicular to the optical axis direction are different from each other.

4. The imaging device according to claim 1, wherein the relay board includes a plurality of dummy pins that are positioned on the substrate, the dummy pins being not electrically connected with the sensor electrodes and/or the cores.

5. The imaging device according to claim 4, wherein the dummy pins stand around an electronic component having a high heat radiation among the one or more electronic components mounted on the substrate.

6. The imaging device according to claim 4, wherein the dummy pins are provided to be exposed at a side face, which is parallel to the optical axis direction, among side faces of the body part.

7. The imaging device according to claim 4, wherein the dummy pins stand on a pattern drawn from an electronic component having a high heat radiation among the one or more electronic components mounted on the substrate.

8. An endoscope system to be inserted into a living body to image an inside of the living body, the endoscope system comprising:
an endoscope including the imaging device according to claim 1 at a distal end portion.

9. A method of manufacturing the imaging device according to claim 1, the method comprising:
a mother board producing step of producing a mother board of the relay board;
a first connection step of connecting sensor electrodes formed on a wafer with substrate electrodes formed on a front face of the mother board, a plurality of light receiving units and the sensor electrodes being formed on the wafer;
a second connection step of connecting the cores of the cables with cable electrodes formed on a rear face of the mother board, the cores being exposed on a connection plane of a large cable assembly including the cable assemblies connected by the cable fixing member; and
a dicing step of dicing the wafer, the mother board, and the large cable assembly in parallel with an optical axis direction of the light receiving units to form the imaging device.
